(19) United States
(12) Patent Application Publication
(10) Pub. No.: US 2006/0073067 A1
(43) Pub. Date: Apr. 6, 2006

(54) Peracid-based large-area decontamination

(75) Inventors: Amanda S. Schilling, Fredericksburg, VA (US); Richard C. Hodge, King George, VA (US)

Correspondence Address: NAVAL SURFACE WARFARE CENTER, DAHLGREN DIVISION;OFFICE OF COUNSEL, CODE XDC1, 17320 DAHLGREN ROAD, DAHLGREN, VA, 22448-5110, US

(73) Assignee: Government of the United States of America as represented by the Secretary of the Navy

(21) Appl. No.: 10/963,827

(22) Filed: Oct. 6, 2004

(51) Int. Cl. A61L 2/18

(52) U.S. Cl. 422/22

(57) ABSTRACT

A method, and product, for large-scale decontamination uses a stable, solid peracid compound, such as acetyl peroxyborate.

### STATEMENT REGARDING FEDERALLY SPONSORED RESEARCH OR DEVELOPMENT

The invention described herein may be manufactured and used by or for the government of the United States of America for governmental purposes without the payment of any royalties thereon or therefor.

### BACKGROUND OF THE INVENTION

1. Field of the Invention

The present invention provides large-scale decontamination using a stable, solid peracid compound.

2. Brief Description of the Related Art

Biological agents may present a health hazard and be difficult to eradicate. For example, Bacillus anthracis and other endospore forming bacteria are highly resistant to heat, radiation, chemical treatment and other environmental extremes when in the dormant spore form. When present over a large area or volume, these biological are especially problematic to decontaminate.

The decontamination of bacterial endospores typically necessitates the use of harsh, toxic and corrosive chemicals. Several compositions are employed for decontamination of biologicals, such as the highly corrosive, flammable and toxic DS2 (Decontamination Solution 2) previously employed by the Department of Defense (DoD). Further drawbacks to DS2 included its incompatibility with some military materials, health hazards and associated environmental concerns. Several other decontamination solutions are available for use in decontamination large areas or surfaces. Aldehydes, for example, are known as effective biocides, with sporicidal activity. Glutaraldehyde, in particular, is often selected due to its activity against a wide range of microbes and its noncorrosive properties. The drawback to glutaraldehyde is that it produces toxic fumes of carbon monoxide. Furthermore, laboratory experiments have shown glutaraldehyde to have mutagenic effects. Formaldehyde also is problematic, with the most serious problem stemming from the vapors that may present a carcinogenic risk. Peracetic acid has been described as an excellent bactericide, fungicide, and sporicide (see Baldry, M. G. C., et al., Journal of Applied Bacteriology, 1983, 54, 417-423). This compound is corrosive to iron, zinc, and copper and alloys containing these metals such as plain steel, galvanized iron, brass, and bronzes; however, the corrosivity is dependent on the concentration of the oxidizer in the solution applied and the contact time. Commercial solutions of peracetic acid are usually equilibrium solutions of hydrogen peroxide, acetic acid, water, and peracetic acid.

Chlorine and hydrogen peroxide compounds are two other choices for disinfection of bacterial endospores. High Test Hypochlorite (HTH) is often selected for decontamination of biologicals due to its rapid action and nonflammable properties. However, the solution is known for its instability and corrosiveness. Hydrogen peroxide concentrations used for disinfection may irritate the eyes, skin and mucous membranes. Additionally, hydrogen peroxide is corrosive at high concentrations and under conditions of high heat or pressure may explode.

The drawbacks of the above compounds may lead to the selection of other biocidal solutions. Alcohols are essentially ineffective against endospore forming bacteria, although isopropyl and ethyl alcohol are known for their antimicrobial activity for vegetative cells, viruses, and fungi. Alcohols are suitable for topical application, and, after repeated use, may damage rubber or plastics; however, the flammability of alcohols is problematic.

Phenols are known to have even less antimicrobial and antivirucidal activity. Phenolic compounds may irritate skin and other tissues. Quaternary ammonium compounds are cationic, surface active compounds that are not usually sporicidal. The activity of such compounds against spores, tuberculosis, and hydrophilic viruses is very poor. Furthermore, quaternary ammonium compounds may irritate the skin and eyes, and have some toxicity concerns.

There is a need in the art to provide environmentally-safe large-scale decontamination. The present invention addresses this and other needs.

### SUMMARY OF THE INVENTION

The present invention includes a method for large-scale decontamination that uses an effective amount of a stable, solid peracid compound or a stable, solid source of a peracid compound to contact a contaminated surface. Peracetic acid from acetyl peroxyborate is preferred.

The present invention also includes the resultant decontaminated surface that has been decontaminated by contacting it with the stable, solid peracid or peracid source.

### BRIEF DESCRIPTION OF THE DRAWINGS

FIG. 1 shows the surviving colony forming units per milliliter (CFU/ml) of Bacillus globigii after 15 minutes exposure to 1 mg/ml PAB;

FIG. 2 shows the surviving colony forming units per milliliter (CFU/ml) of Bacillus anthracis Vollum 1B after 15 minutes exposure to 65 mg/ml PAB; and,

FIG. 3 shows the surviving colony forming units per milliliter (CFU/ml) of Bacillus anthracis Vollum 1B after 15 minutes exposure to 65 mg/ml PAB.

### DETAILED DESCRIPTION OF THE PREFERRED EMBODIMENT

The present invention provides a method for large-scale decontamination using a stable, solid peracid or a stable, solid source of a peracid (herein referred to as “peracid compound”), such as acetyl peroxyborate, and derivatives thereof. In a preferred embodiment, a stable, solid form of the peracetic acid (PAA) is used to readily, effectively and efficiently decontaminate biological and other like contaminants present in voluminous amounts and/or substantial areas, generally referred to herein as large-area decontamination.

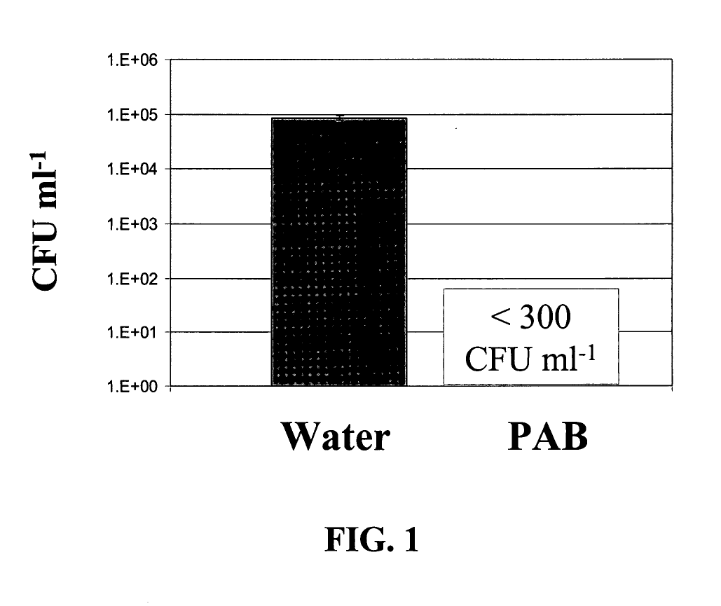

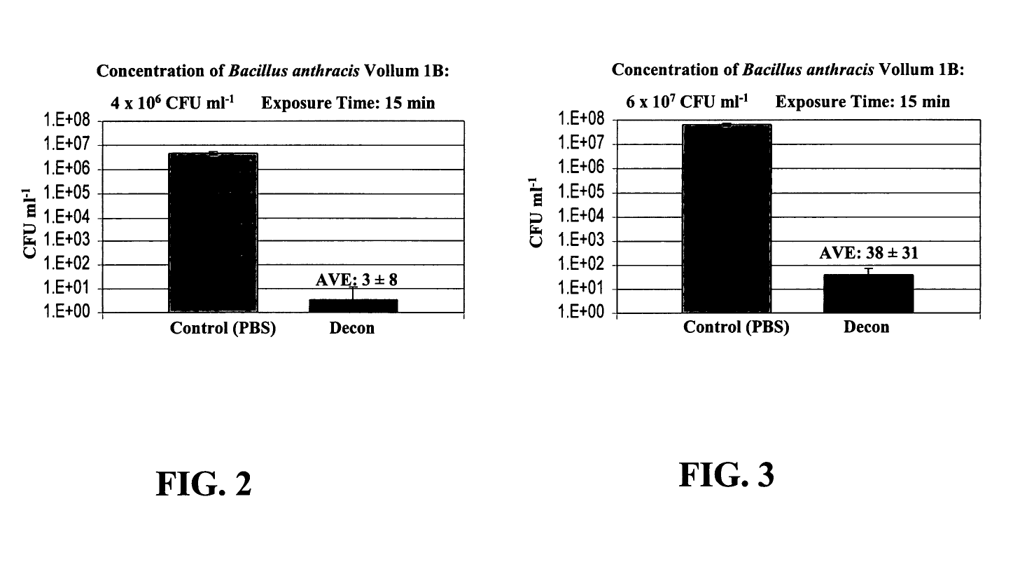

Peracids, such as that released from acetyl peroxyborate, constitute an efficient decontaminant for biological agents, including endospore forming bacteria. As seen in FIGS. 1, 2 and 3, the use of acetyl peroxyborate (PAB) is shown as an effective decontaminant. FIG. 1 shows the surviving colony forming units per milliliter (CFU/ml) of Bacillus globigii after 15 minutes exposure to 1 mg/ml PAB. The starting concentration of Bacillus globigii was 8.5×10⁴ CFU/ml with a final concentration of <300 CFU/ml. FIG. 2 shows the surviving colony forming units per milliliter (CFU/ml) of Bacillus anthracis Vollum 1B after 15 minutes exposure to 65 mg/ml PAB. The starting concentration of Bacillus anthracis was 4×10⁶ CFU/ml. FIG. 3 shows the surviving colony forming units per milliliter (CFU/ml) of Bacillus anthracis Vollum 1B after 15 minutes exposure to 65 mg/ml PAB. The starting concentration of Bacillus anthracis was 6×10⁷ CFU/ml.

Peracid compounds of the present invention include derivatives of organic acids having one or more directly linked pairs of oxygen atoms that provide biocidal properties. Selection of the appropriate peracid compound is determinable by those skilled in the art in light of the disclosure herein. Representative peracids and/or stable sources of peracids of the present invention include, for example without limitation, acetyl peroxyborate, peroxyacetic acid, peroxynonanoic acid, and peroxybenzoic acid and other like compounds and derivatives thereof. Preferably, the stable, solid peracid compound includes acetyl peroxyborate and derivatives of acetyl peroxyborate, including coated, admixed and other like compounds. Stable compounds of the present invention are defined as those compounds that, when in solid form, are not explosive, detonatable or shock sensitive or otherwise sensitive to, or that rapidly degrade during, storage, transport or handling. Solid compositions include those forms of the peracids that retain granular consistencies under ambient temperature and humidity conditions, such as 0° C. to 50° C. and 0% to 70% relative humidity. Stable, solid acetyl peroxyborate compounds are known, such as that disclosed in U.S. Pat. No. 5,424,079 to Yu, and U.S. Pat. No. 5,462,692 and U.S. Pat. No. 6,086,785, both to Roesler et al., the disclosures of which are herein incorporated by reference with regard to such compounds.

In one preferred embodiment, the present invention includes a stable, solid acetyl peroxyborate compound and water. The desired amount of stable, solid acetyl peroxyborate compound is measured out and then water is added. This solution may then be applied to any surface where decontamination of biological agents is necessary. As such, advantageously the present invention provides an effective decontaminant with minimal components, having low corrosivity and being environmentally friendly. Perferably, only water and the acetyl peroxyborate compound components are present. The solid acetyl peroxyborate compound is lightweight and stable, reducing the logistical concerns associated with current military and commercial decontaminants. Use of the solid, stable acetyl peroxyborate compound of the present invention eliminates stability and logistical problems inherent with liquid compositions. The need for small quantities of the acetyl peroxyborate as effective anti-biological agents greatly reduces the corrosion hazard on various materials.

Use of the peracid-based decontaminant may include application of the peracid compound onto the biological contaminant prior to application of the water, or the peracid compound may be mixed with the water prior to application onto the biological contaminant. An appropriate amount of peracid compound is used for decontamination of the surface, which may be varied by the sized of the surface, amount of contamination, amount of solubilizing water used, and other like factors. The concentration of the peracid compound or the volume of water may be varied, in light of such factors as the type of biological hazard, area to be decontaminated, amount of peracid compound available, operational status, time available for decontamination, with these and other like factors determinable by those skilled in the art of decontamination. Preferably, the acetyl peroxyborate is present in the water, when solubilized, in an amount of from about 0.0005 g/liter or greater, more preferably from about 0.0005 g/liter to about the saturation point of the amount of solubilizing water, and most preferably from about 0.065 g/liter to about 0.130 g/liter. When used in a dry state, the acetyl peroxyborate is preferably present in an amount of from about 0.0005 g/square meter or greater, more preferably from about 0.0005 g/square meter to about 0.130 g/square meter, and most preferably from about 0.065 g/square meter to about 0.10 g/square meter.

In practice, the method of the present invention includes contacting a contaminated surface with an effective amount of acetyl peroxyborate. The acetyl peroxyborate may include a non-solubilized, or dry, form of the compound. Preferably, the acetyl peroxyborate is solubilized prior to contact with the contaminated surface to provide a more uniform contact of the acetyl peroxyborate across the entire contaminated surface. Solubilization of the acetyl peroxyborate may occur prior to or after the acetyl peroxyborate is placed on the contaminated surface, such as combining the acetyl peroxyborate and water in a container and applying the combined product onto the contaminant, application of water onto the contaminated surface followed by the application of the acetyl peroxyborate therein, or application of the acetyl peroxyborate onto the contaminated surface followed by the application of the water.

With the application of the solubilized peracid compound, the solution may be allowed to dry on the contacted contaminated surface. The residue is then preferably rinsed with water to remove the decontaminated composition incorporating the original contaminant from the surface.

Solubilizing the acetyl peroxyborate with water may also be accomplished using one or more mixers, containing the acetyl peroxyborate and water, prior to application of the acetyl peroxyborate on the contaminant. Mixing routines may include, for example, adding the water into a container containing the peracid compound, adding the peracid compound into a container containing the water or mixing the components in a third container. Once combined, the solubilized acetyl peroxyborate may be applied to the contaminant by any appropriate means for a given area or article, as determinable by those skilled in the art. Application includes washing application systems, sprayers, brushes, mops and other like applicators, useful for a given area or article. Preferably, the application of the solubilized acetyl peroxyborate occurs in a manner that allows for the most effective concentration of the acetyl peroxyborate to contact contaminant spores for a sufficient period of time for effective decontamination. The area may include the interior and/or exterior of buildings, floors and hallways, flight decks, buildings, vehicle surfaces, maritime vessels such as cargo ships, aircraft carriers and other warships, and other like structures. Articles include objects and devices such as mechanical devices, industrial equipment, vehicles, and other large-scale object surfaces, and the like. Surfaces may include vertical or horizontal hard surfaces, equipment, textiles, hazmat protective gear and and other clothing and equipment, etc. Additionally, contaminated surfaces may include personnel. Preferably contaminated surfaces are contacted with the solubilized acetyl peroxyborate by spraying the surface with the solubilized acetyl peroxyborate, such as through fire hose connections or wash down systems on board naval vessels.

Once contacted with the peracid compound, the contaminated surface may be rinsed, preferably with water. Sources of water may be used as advantageously available, with selection of these water sources determinable by those skilled in the art of large-scale decontamination. Applications of the peracid compound may be made, as desired, for more complete decontamination, or to address additional contamination that has occurred after the original application of the peracid.

Advantageously, the method of the present invention provides an isolated solid for decontaminant use. In a most preferred embodiment, the solid acetyl peroxyborate compound is located at individual stations on board a warship. As a solid, the decontaminant has minimal impact on the warship's buoyancy characteristics, superstructure weight distributions, tainting of the fresh water supply, etc., while being readily available for decontamination. In the event of biological contamination, the solid acetyl peroxyborate may be immediately dispersed onto the deck and/or bulkheads, or other parts of the superstructure of the warship in solid form, with area previously or later wetted with sea or fresh water. Alternatively, the solid acetyl peroxyborate may be contained within part of a flushing system that injects the water through the contained solid acetyl peroxyborate that is then sprayed onto the contaminated superstructure. With the application of the acetyl peroxyborate onto the superstructure of the warship, the contaminated area may be agitated, preferably in a mechanical manner, to thoroughly dispense the acetyl peroxyborate into the biological contaminant. Application of the peracid compound may include any desirable means for a given area or article, as determinable by those skilled in the art. Application includes washing application systems, sprayers, brushes, mops and other like applicators, useful for a given area or article. Preferably, the application of the peracid compound occurs in a manner that allows for the most effective concentration of the peracid compound to contact contaminant spores for a sufficient period of time for effective decontamination.

Additional compositions may be added to the peracid compound/water mixture of the present invention, as desired. These compositions are selected for particular purposes, using a broad range of selection criteria, such as non-interference with the decontaminating functionality of the peracid compound, ability to desiccate the peracid compound, decontaminating properties inherent within the compositions, color indicators, corrosion inhibitors and other characteristics useful in the application of a decontaminating composition.

The present invention is applicable for decontamination of biological contaminants, such as biological spores, particularly biological spores that comprise bacterial endospores. As used herein, the terms “spores”, “biological spores”, “spore populations” and similar terminology, refer to contaminant spores that create a hazard, threat, nuisance, etc. by their presence in an environment, on a surface, in food, etc. Typical spores decontaminated by the present invention include, for example, endospores, such as those belonging to the genus Bacillus and Clostridium. Representative endospore populations include almost all Bacillus and Clostridium species, including, but not limited to Bacillus subtilis, Bacillus anthracis and Bacillus globigii. The present invention may also be applied toward the decontamination of fungal endospores such as Helminthosporium viruses such as Orthopox, toxins such as ricin, and other classes of bacteria such as Salmonella.

Effectiveness of the methodology of the present invention occurs with increases of biological spore “kills” with the use of the acidic environment plus heat over non-use of such conditions. Preferably, an effective kill is dependent on the original number of spores within a contamination, such as a 90% effectiveness (kill) against a concentration of 10³ spores/ml, and more preferably an effectiveness of 90% against a concentration of 10⁸ spores/ml, with a most preferred decontamination of from about three or more logarithmic reductions of live spores. Most preferably, the decontamination reduces the spore concentration to a level that renders the once hazardous contaminated area or surface no longer hazardous. Effective biological spore decontamination of these spores rids a contaminated space or object of the immediate hazard occasioned by the spore presence. Spores are neutralized when they are rendered harmless, i.e., no longer hazardous, to a particular living organism, particularly a human. Depending on the circumstances, spore decontamination may be desirable against spores that affect other mammals, animals or plants. Decontamination applications non-exclusively include decontamination of endospore-forming bacteria and fungi, non-spore forming bacteria, viruses and toxins in military, medical, industry, agriculture, and household domains, particularly in the event of accidental contamination or terrorist attack. Representative localities that might benefit from the decontamination regimen of the present invention for reduction of spore populations include hospitals, veterinary clinics, farms, dairies, meat processing facilities, hide processing facilities, ships, buildings, houses, automobiles and other like contaminated surfaces and/or areas.

### EXAMPLE 1

A hospital hallway, having dimensions of 150 feet by 20 feet, is contaminated with biological spores. An empty 55-gallon drum, with a hand pump, is placed near the hallway. Five kilograms of solid, stable acetyl peroxyborate is poured into the 55-gallon drum and 50 gallon of fresh water are added, and the acetyl peroxyborate and water are mixed. The hand pump is used to pump the acetyl peroxyborate onto the floor of the hallway. After one hour, mops are used by personnel in hazmat suits to scrub the floor and side walls. A vacuum device, effective for trapping the solubilized acetyl peroxyborate, removes liquid and decontaminated spores from the floor.

### EXAMPLE 2

The procedure of Example 1 is performed, and repeated in the hospital hallway.

### EXAMPLE 3

A building, having 400,000 square feet of surface area, is contaminated with biological spores. A pump truck having a capacity of 5000 gallons is driven to the area of the building. The pump truck contains 500 kilograms of acetyl peroxyborate dispersed in 4500 gallons of fresh water. The acetyl peroxyborate/water mixture is pumped on the outside and inside of the building, where personnel in hazmat suits use mops to further disperse the mixture into the contaminant. Excess liquid is removed from the building, which is dried using fans.

### EXAMPLE 4

During military combat operations, an aircraft carrier is contaminated with biological spores. Holding stations located around the ship contain solid acetyl peroxyborate. The solid acetyl peroxyborate is poured onto the contaminated decks and bulkheads of the ship, and after 15 minutes, is sprayed with salt water using fire hoses. Another batch of solid acetyl peroxyborate is applied to the wet deck and bulkheads. After 30 minutes the decks and bulkheads are sprayed again.

The foregoing summary, description, and examples of the present invention are not intended to be limiting, but are only exemplary of the inventive features which are defined in the claims.

What is claimed is:

1. A method for large-scale decontamination, comprising the steps of:
providing an amount of stable, solid peracid compound effective for decontaminating a contaminated surface; and,
contacting the contaminated surface with the stable, solid peracid compound effective for decontamination thereof.

2. The method of claim 1, wherein the stable, solid peracid compound comprises a stable, solid acetyl peroxyborate compound.

3. The method of claim 1, further comprising the step of solubilizing the stable, solid peracid compound in water prior to contacting the contaminated surface.

4. The method of claim 3, wherein the stable, solid peracid compound is placed on the contaminated surface prior to the step of solubilizing the stable, solid peracid compound in water.

5. The method of claim 3, wherein the step of solubilizing the stable, solid peracid compound in water comprises the application of water onto the contaminated surface and then applying the stable, solid peracid compound therein.

6. The method of claim 1, further comprising the step of rinsing the contacted contaminated surface with water.

7. The method of claim 6, wherein the step of rinsing comprises the application of salt water.

8. The method of claim 3, wherein the solubilized stable, solid peracid compound is allowed to dry on the contacted contaminated surface.

9. The method of claim 3, wherein the step of providing an amount of stable, solid peracid compound comprises an amount of from about 0.0005 g/liter or greater.

10. The method of claim 9, wherein the stable, solid peracid compound comprises an amount of from about 0.0005 g/liter to about the saturation point of the amount of solubilizing water.

11. The method of claim 10, wherein the stable, solid peracid compound comprises an amount of from about 0.065 g/liter to about 0.130 g/liter.

12. The method of claim 3, wherein the step of solubilizing the stable, solid peracid compound in water comprises adding the water into a container containing the stable, solid peracid compound.

13. The method of claim 1, wherein the step of solubilizing the stable, solid peracid compound in water comprises adding the stable, solid peracid compound into a container containing the water.

14. The method of claim 3, wherein the step of contacting the contaminated surface with the solubilized acetyl peroxyborate comprises spraying the surface with the solubilized acetyl peroxyborate.

15. The method of claim 1, wherein the step of contacting the contaminated surface with the stable, solid peracid compound comprises mechanical agitation.

16. The method of claim 1, further comprising an additional step of re-contacting the contaminated surface with the stable, solid peracid compound effective for decontamination thereof.

17. The method of claim 6, further comprising an additional step of re-contacting the contaminated surface with the stable, solid peracid compound effective for decontamination thereof.

18. A large-scale decontaminated surface product comprising the method of claim 1.

19. The product of claim 18, wherein the large-scale decontaminated surface product comprises the structure of a warship.

20. The product of claim 18, wherein the large-scale decontaminated surface product comprises household surfaces.
